(19) United States
(12) Patent Application Publication
(10) Pub. No.: US 2018/0362488 A1
(43) Pub. Date: Dec. 20, 2018

(54) STEREOISOMER OF FLOCOUMAFEN, COMPOSITION AND RODENTICIDE BAIT COMPRISING SAME, AND METHOD FOR CONTROLLING TARGET RODENT PESTS

(71) Applicants: LIPHATECH, Pont-Du-Casse (FR); INSTITUT ENSEIGNEMENT SUPERIEUR ET RECHERCHE EN ALIMENTATION SANTE ANIMALE SCIENCES AGRONOMIQUES, Marcy L'Etoile (FR)

(72) Inventors: Hervé CARUEL, Moncaut (FR); Etienne BENOIT, Lyon (FR); Isabelle FOUREL, Miribel (FR); Virginie LATTARD, Lyon (FR)

(21) Appl. No.: 16/061,092

(22) PCT Filed: Dec. 6, 2016
(86) PCT No.: PCT/EP2016/079863

§ 371 Date: Jun. 11, 2018

(30) Foreign Application Priority Data
Dec. 11, 2015 (FR) 1562167

(51) Int. Cl. C07D 311/56; A01N 43/16; A01N 25/00

(52) U.S. Cl. CPC C07D 311/56; C07B 2200/07; A01N 25/004; A01N 43/16

(57) ABSTRACT

Disclosed is a configurational stereoisomer, referred to as enantiomer E₂, of flocoumafen, the enantiomer E₂ having, as determined by the chromatographic analysis of a flocoumafen composition including four configurational stereoisomers of flocoumafen, carried out under conditions described hereinafter, a retention time t₂ with a value such that t₁<t₂<t₃<t₄; t₁, t₃ and t₄ representing the retention times of the configurational stereoisomers of flocoumafen different from the enantiomer E₂, the chromatographic analysis being carried out at a temperature of 23.5° C.

The invention relates to a configurational stereoisomer of flocoumafen, to a composition and a rodenticidal bait comprising such a configurational stereoisomer and to a process for controlling target rodent pests. The invention also relates to a process for obtaining such a configurational stereoisomer of flocoumafen. The invention thus relates to the technical field of controlling populations of target rodent pests.

It is known practice to use poisons in the form of rodenticidal baits against target rodent pests. WO 2005/072524 discloses a rodenticidal bait comprising a proportion of 50 ppm of flocoumafen in the bait and a proportion of 40 ppm of fipronil.

Such a bait is liable to be consumed by animals other than target rodent pests when it is made available to target rodent pests. It may be consumed directly (primary consumption) by domestic animals or pets. It may also be consumed accidentally by humans. Such consumption may induce poisoning, which may be lethal, of these domestic animals, pets or humans.

In addition, a fraction of the flocoumafen of these rodenticidal baits may be ingested (secondary consumption) by animals—especially by birds—which prey or carrion-feed on rodent pests and especially weakened target rodent pests that have consumed such a rodenticidal bait. This secondary consumption is liable in the long term to result in the death of these predatory or carrion-feeding animals, which may be animals—especially birds—belonging to protected species.

The invention is thus directed towards overcoming these drawbacks by proposing a configurational stereoisomer of flocoumafen, a composition and a rodenticidal bait comprising such a configurational stereoisomer and a process for controlling target rodent pests, which are effective for controlling the populations of target rodent pests and can also limit the risks of poisoning of non-target animals—especially domestic or reared animals, pets or humans—which accidentally consume such a rodenticidal bait.

The invention is also directed towards overcoming these drawbacks by proposing a configurational stereoisomer of flocoumafen, a composition and a rodenticidal bait comprising such a configurational stereoisomer and a process for controlling target rodent pests, which are effective for controlling populations of target rodent pests and can also limit the risks of secondary poisoning of wild animals—for example foxes or birds—which prey on weakened target rodent pests that have consumed the rodenticidal bait or of wild animals which carrion-feed on the corpses of poisoned target rodent pests.

The invention is also directed towards proposing a configurational stereoisomer of flocoumafen, a composition and a rodenticidal bait comprising such a configurational stereoisomer and a process for controlling target rodent pests, the use of which is in accordance with the rules of good practice, especially with respect to the protection of birds, and in particular birds of prey.

The invention is also directed towards proposing a configurational stereoisomer of flocoumafen, a composition and a rodenticidal bait comprising such a configurational stereoisomer and a process for controlling target rodent pests, which are kind to the environment, human health and non-target animals, especially birds, and in particular birds of prey.

The invention is also directed towards proposing a configurational stereoisomer of flocoumafen, a composition and a rodenticidal bait comprising such a configurational stereoisomer and a process for controlling target rodent pests, which are able to be used for controlling target rodent pests that are resistant to known baits for controlling target rodent pests.

The invention is thus also directed towards proposing an alternative to known rodenticidal substances and baits.

To do this, the invention relates to a configurational stereoisomer, named enantiomer E₂, of flocoumafen, said enantiomer E₂ having, by chromatographic analysis of a flocoumafen composition comprising four configurational stereoisomers of flocoumafen performed under the conditions described below, a retention time t₂ having a value such that t₁<t₂<t₃<t₄; t₁, t₃ and t₄ representing the retention times of the configurational stereoisomers of flocoumafen different from said enantiomer E₂, the chromatographic analysis being performed at a temperature of 23.5° C. and under the following conditions:

on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;

using, as liquid mobile phase, a mixture formed from acetonitrile (A) and water comprising 0.1% by volume of formic acid (B), with an AB volume ratio of 92/8 and with a flow rate of the liquid mobile phase in the chromatography column of 0.25 mL/minute;

by injection into the chromatography column of a volume of 1 μL of flocoumafen composition at a concentration of 1 μg of flocoumafen per millilitre of acetonitrile.

Throughout the text:

the term “flocoumafen” denotes the compound of formula 3-[4-(4-trifluoromethylbenzyloxy)phenyl-4-yl]-1-(4-hydroxycoumarin-3-yl)-1,2,3,4-tetrahydronaphthalene or 4-hydroxy-3-[1,2,3,4-tetrahydro-3-[4-[[4-(trifluoromethyl)phenyl]methoxy]phenyl]-1-naphthalenyl]-2H-1-benzopyran-2-one, or 4-hydroxy-3-[1,2,3,4-tetrahydro-3-[4-(4-trifluoromethylbenzyloxy)phenyl]-1-naphthyl]coumarin of formula (I) below:

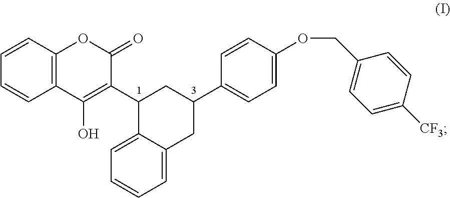 [SMILES: O=c1oc2ccccc2c(O)c1C1CC(c2ccc(OCc3ccc(C(F)(F)F)cc3)cc2)Cc2ccccc21]

in which are represented the numbers of carbons 1 and 3 of the 1,2,3,4-tetrahydronaphthalene group;

the term “stereoisomers” denotes isomers of the same semi-structural formula, but in which the relative position of the atoms differs in space. The term “configurational stereoisomers” denotes stereoisomers for which conversion from one to the other of these configurational stereoisomers requires the cleavage/reformation of an interatomic covalent bond. Thus, the term “configurational stereoisomers” denotes stereoisomers which are not conformational isomers (or “rotamers”, for which conversion from one to the other of the conformational isomers is accompanied only by rotation of a part of the molecule about the axis of a σ (sigma) bond formed by axial orbital overlap);

the term “amount” means a molar amount, a mass amount or a volume amount. The proportions are thus proportions of a molar amount relative to a molar amount, of a mass amount relative to a mass amount, or of a volume amount relative to a volume amount;

the term “substantially” indicates, in the usual manner, that a structural or functional characteristic should not be taken as marking an abrupt discontinuity, which would have no physical meaning, but covers not only this structure or this function, but also slight variations of this structure or of this function which produce, in the technical context under consideration, an effect of the same nature, or else of the same degree;

the expressions “high-pressure liquid chromatography” or “high-performance liquid chromatography” (HPLC) denote “HPLC” chromatography or “High Performance Liquid Chromatography”; and

the term “retention time” denotes the time, measured at the top of the peak in the chromatogram, for which a compound is retained on the chromatography column.

The invention thus relates to said enantiomer E₂ in isolated form, which has the property of being able to be eluted second, under the chromatography conditions described above, relative to the four configurational stereoisomers of flocoumafen.

The inventors have observed that analysis of flocoumafen by high-pressure liquid chromatography under the conditions described above reveals four signals or peaks corresponding to four compounds of the same chemical structural formula and corresponding to formula (I) of flocoumafen.

They determined, by analysis of flocoumafen preparations comprising variable proportions of the two diastereoisomers of flocoumafen, that:

the compound corresponding to the signal with a retention time t₁ having a value of the order of 4.5 minutes is one enantiomer, named enantiomer E₁, of one of the two diastereoisomers, named diastereoisomer D₁₄, of flocoumafen;

the compound corresponding to the signal with a retention time t₂ having a value of the order of 6.2 minutes is one enantiomer, named enantiomer E₂, of the other diastereoisomer, named diastereoisomer D₂₃, of flocoumafen, different from said diastereoisomer D_{1,4};

the compound corresponding to the signal with a retention time t₃ having a value of the order of 6.8 minutes is the other enantiomer, named enantiomer E₃, of said diastereoisomer D_{2,3}, different from said enantiomer E₂; and

the compound corresponding to the signal with a retention time t₄ having a value of the order of 9.3 minutes is the other enantiomer, named enantiomer E₄, of said diastereoisomer D_{1,4}, different from said enantiomer E₁.

The retention time values t₁, t₂, t₃ and t₄ are liable to vary, especially with the temperature of the chromatography column. However, under these chromatographic conditions, the order of elution of the flocoumafen enantiomers remains unchanged.

One of the two diastereoisomers of flocoumafen is a configurational stereoisomer of flocoumafen in which carbons 1 and 3 of the 1,2,3,4-tetrahydronaphthalene group of flocoumafen have the same absolute configuration and the other of the two diastereoisomers of flocoumafen is a configurational stereoisomer of flocoumafen in which carbons 1 and 3 of the 1,2,3,4-tetrahydronaphthalene group of flocoumafen have different absolute configurations, the absolute configurations being determined according to the Cahn-Ingold-Prelog sequential priority and nomenclature rules.

The inventors performed such a separation of the configurational stereoisomers, i.e. of the enantiomers of the two diastereoisomers, of flocoumafen by high-pressure liquid chromatography on a LUX Cellulose-4 chiral column (00F-4490-B0, Phenomenex, Le Pecq, France). Where appropriate, it is possible successively to perform several high-pressure liquid chromatography steps on a chiral column for the purposes of obtaining the desired amount of said enantiomer E₂ in the desired purity. It is also possible to perform such a separation by high-pressure liquid chromatography on a preparative chiral column of larger dimensions—especially on a chiral column with an inside diameter of greater than 2 mm—and in which the stationary phase has a particle size of greater than 3 They obtained said enantiomer E₂ purified and separated from enantiomer E₃ of said diastereoisomer D₂₃ of flocoumafen and from the enantiomers E₁ and E₄ of said diastereoisomer D_{1,4} of flocoumafen by removing the mobile phase from the collected fraction containing said enantiomer E₂.

A method for separating the enantiomers of said diastereoisomer D₂₃ of flocoumafen was not previously known. Under these experimental conditions, the enantiomers (E₂ and E₃) of said diastereoisomer D₂₃ are separated and purified by high-pressure liquid chromatography. Under these chromatographic conditions, the enantiomers (E₁ and E₄) of said diastereoisomer D_{1,4} of flocoumafen are also separated.

The invention thus relates to said enantiomer E₂ of said diastereoisomer D₂₃ which is the less retained (which has the shorter retention time) of the two enantiomers of said diastereoisomer D₂₃ separated by chromatography under the abovementioned conditions.

The invention thus relates to said enantiomer E₂ separated from enantiomer E₃ of said diastereoisomer D₂₃ and separated from enantiomer E₁ and from enantiomer E₄ of said diastereoisomer D_{1,4}.

The invention also relates to a chromatographic process for separating the configurational stereoisomers—especially said enantiomers E₁ and E₄ of said diastereoisomer D_{1,4} and said enantiomers E₂ and E₃ of said diastereoisomer D₂₃. The invention also relates to a chromatographic process for obtaining said enantiomer E₂ according to the invention.

The invention thus relates to such a chromatographic process for obtaining said enantiomer E₂ according to the invention, in which:

a high-pressure liquid chromatography column of dimensions 150×2 mm, and comprising a chiral stationary phase constituted of particles of tris(4-chloro-3-methylphenyl carbamate) cellulose, is chosen, said particles having a mean size of 3 μm and having a mean pore size of 1000 Å;

a mixture formed from acetonitrile (A) and water comprising 0.1% by volume of formic acid (B), with an A/B volume ratio of 92/8 and with a flow rate of the liquid mobile phase in the chromatography column of 0.25 mL/minute, is chosen as liquid mobile phase;

separation of the configurational stereoisomers of flocoumafen is performed at room temperature, during which:

a liquid composition comprising said enantiomer E₂ is introduced into the top of the chromatography column; and then

the liquid composition is entrained with the mobile phase in the chromatography column under conditions suitable for separating the configurational stereoisomers of flocoumafen, and a fraction of the mobile phase comprising said enantiomer E₂ with a retention time t₂ having a value such that t₁<t₂<t₃<t₄; t₁, t₃ and t₄ representing the retention times of each of the configurational stereoisomers of flocoumafen different from said enantiomer E₂, is collected separately from said enantiomer E₃ of retention time t₃; and

the liquid mobile phase of said fraction is removed so as to obtain said enantiomer E₂.

The invention also relates to said enantiomer E₂ obtained via a process according to the invention.

The invention also relates to a composition comprising said enantiomer E₂ according to the invention, with the exclusion of a racemic mixture of said enantiomer E₂ and of said enantiomer E₃. The invention thus relates to a composition comprising said enantiomer E₂ according to the invention, with the exclusion of a racemic mixture of the two enantiomers E₂ and E₃ of said diastereoisomer D_{2,3}.

The invention thus relates to a composition comprising a configurational stereoisomer, named enantiomer E₂, of flocoumafen, with the exclusion of a racemic mixture of said enantiomer E₂ and of a configurational stereoisomer, named enantiomer E₃, of flocoumafen;

said enantiomer E₂ having, by chromatographic analysis of a flocoumafen composition comprising four configurational stereoisomers of flocoumafen performed under the conditions described below, a retention time t₂;

said enantiomer E₃ having, by chromatographic analysis of a flocoumafen composition comprising four configurational stereoisomers of flocoumafen performed under these same conditions, a retention time t₃; t₂ and t₃ being values such that t₁<t₂<t₃<t₄; t₁ and t₄ representing the retention times of each of the configurational stereoisomers of flocoumafen different from said enantiomer E₂ and from said enantiomer E₃, the chromatographic analysis being performed at a temperature of 23.5° C. and under the following conditions:

on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;

using, as liquid mobile phase, a mixture formed from acetonitrile (A) and water comprising 0.1% by volume of formic acid (B), with an AB volume ratio of 92/8 and with a flow rate of the liquid mobile phase in the chromatography column of 0.25 mL/minute;

by injection into the chromatography column of a volume of 1 μL of flocoumafen composition at a concentration of 1 μg of flocoumafen per millilitre of acetonitrile.

The invention thus relates to such a composition comprising said enantiomer E₂, with the exclusion of a racemic mixture of said enantiomer E₂ and of said enantiomer E₃, i.e. with the exclusion of a composition in which said enantiomer E₂ and said enantiomer E₃ are in equimolar mixture and not optically active.

Said enantiomer E₂ and said enantiomer E₃ of any composition comprising flocoumafen are assayed by chromatographic analysis and separation using a chiral stationary phase and a liquid mobile phase as described above for the analysis of the configurational stereoisomers of flocoumafen, by performing quantitative detection of the configurational stereoisomers of flocoumafen at the outlet of the separating column, for example by absorption photometry or spectrophotometry, adjusting the flocoumafen concentration and the injection volume for the purposes of obtaining optimum detection and measuring the value of the area under the peak for each enantiomer E₂ and E₃. It is also possible to assay said enantiomer E₂ and said enantiomer E₃ of any composition comprising flocoumafen by performing detection by mass spectrometry at the outlet of the separating column.

Advantageously and according to the invention, the amount of said enantiomer E₂ is greater than the amount of said enantiomer E₃ in the composition. In the composition, said diastereoisomer D₂₃ is predominantly in the form of said enantiomer E₂. The composition according to the invention comprises said diastereoisomer D₂₃ predominantly in the form of said enantiomer E₂.

Throughout the text, the term “said diastereoisomer D_{2,3} is predominantly in the form of said enantiomer E₂” means that the ratio of the amount (on a mass, molar or volume basis) of said enantiomer E₂ to the amount (on a corresponding mass, molar or volume basis) of said diastereoisomer D₂₃ (in all its enantiomeric forms) is greater than 50%.

Thus, in particular, in a composition according to the invention:

the ratio of the amount of said enantiomer E₂ to the sum of the amount of said enantiomer E₂ and of the amount of said enantiomer E₃ is greater than 0.5 (greater than 50%);

the ratio of the concentration of said enantiomer E₂ to the sum of the concentration of said enantiomer E₂ and of the concentration of said enantiomer E₃ is greater than 0.5 (greater than 50%); and

the proportion of said enantiomer E₂ in the composition is greater than the proportion of said enantiomer E₃ in the composition.

Advantageously and according to the invention, the composition comprises an amount of said enantiomer E₂ such that the ratio of this amount to the sum of the amount of said enantiomer E₂ and of the amount of said enantiomer E₃ present in the composition is greater than 50%, especially greater than 60%, in particular greater than 70%, more particularly greater than 80%, preferably greater than 90%, more preferentially greater than 95%, particularly preferentially greater than 98%, even more preferentially greater than 99% or about 100%.

In a particular embodiment, advantageously and according to the invention, the composition comprises an amount of said enantiomer E₂ such that the ratio of this amount to the sum of the amount of said enantiomer E₂ and of the amount of said enantiomer E₃ present in the composition is greater than 75%, preferably between 85% and 100% and more preferentially between 90% and 98%.

In another embodiment, advantageously and according to the invention, the composition comprises an amount of said enantiomer E₂ such that the ratio of this amount to the sum of the amount of said enantiomer E₂ and of the amount of said enantiomer E₃ present in the composition is between 98% and 100%.

The composition may also comprise an amount of said enantiomer E₃ such that the ratio of this amount to the sum of the amount of said enantiomer E₂ and of the amount of said enantiomer E₃ is less than 50%, especially less than 25%, preferentially between 0% and 25%, in particular less than 10%.

Advantageously and according to the invention, flocoumafen is predominantly in the form of said enantiomer E₂ in the composition. The composition thus comprises an amount of said enantiomer E₂ such that the ratio of this amount to the amount of flocoumafen in the composition is greater than the ratio of the amount of said enantiomer E₃ to the amount of flocoumafen in the composition and greater than the ratio of the amount of each enantiomer (E₁ and E₄) of said diastereoisomer D_{1,4} to the amount of flocoumafen in the composition.

Thus, in particular, in a composition according to the invention:

the ratio of the amount of said enantiomer E₂ to the amount of flocoumafen is greater than 0.25 (greater than 25%);

the ratio of the amount of said enantiomer E₂ to the sum of the amounts of each enantiomer of said diastereoisomer D_{1,4} and of the amounts of each enantiomer of said diastereoisomer D₂₃ is greater than 0.25 (greater than 25%);

the ratio of the concentration of said enantiomer E₂ in the composition to the concentration of flocoumafen in the composition is greater than 0.25 (greater than 25%);

the proportion of said enantiomer E₂ in the composition is greater than the proportion of each enantiomer of said diastereoisomer D₁₄ in the composition and than the proportion of said enantiomer E₃ in the composition.

Advantageously and according to the invention, the composition comprises an amount of said enantiomer E₂ such that the ratio of this amount to the amount of flocoumafen in the composition is greater than 25%, especially greater than 50%, in particular greater than 70%, more particularly greater than 80%, preferably greater than 90%, particularly preferentially greater than 95%, more preferentially greater than 98%, even more preferentially greater than 99% or about 100%.

In a particular embodiment, advantageously and according to the invention, the composition comprises an amount of said enantiomer E₂ such that the ratio of this amount to the amount of flocoumafen in the composition is greater than 70%, preferably between 80% and 100% and more preferentially between 90% and 100%.

In another embodiment, advantageously and according to the invention, the composition comprises an amount of said enantiomer E₂ such that the ratio of this amount to the amount of flocoumafen in the composition is between 95% and 99%.

In another particular embodiment, advantageously and according to the invention, the composition comprises an amount of said enantiomer E₂ such that the ratio of this amount to the amount of flocoumafen in the composition is greater than 95%.

In another embodiment, advantageously and according to the invention, the composition comprises an amount of said enantiomer E₂ such that the ratio of this amount to the amount of flocoumafen in the composition is between 98% and 100%, limits inclusive.

In another particularly advantageous embodiment according to the invention, the composition comprises an amount of said enantiomer E₂ such that the ratio of this amount to the amount of flocoumafen in the composition is substantially about 100%.

A composition according to the invention may be substantially free of said enantiomer E₃, i.e. said enantiomer E₃ may optionally be present in the composition but only in trace amount. A composition according to the invention may also be substantially free of said diastereoisomer D₁₄, i.e. said diastereoisomer D₁₄ may optionally be present in the composition but only in trace amount.

Advantageously and according to the invention, the composition is in liquid form and comprises a liquid solvent for flocoumafen. It may be a solution of flocoumafen in a solvent for flocoumafen, with the exclusion of a racemic mixture of said enantiomer E₂ and of said enantiomer E₃. It may also be a solution comprising flocoumafen in a solvent for flocoumafen and in which the amount of said enantiomer E₂ is greater than the amount of said enantiomer E₃. It may also be a solution comprising flocoumafen in a solvent for flocoumafen and in which the flocoumafen is predominantly in the form of said enantiomer E₂.

Advantageously and according to the invention, the composition is in solid form. It may also be a solid comprising flocoumafen, with the exclusion of a racemic mixture of said enantiomer E₂ and of said enantiomer E₃. It may also be a solid comprising flocoumafen and in which the amount of said enantiomer E₂ is greater than the amount of said enantiomer E₃. It may also be a solid comprising flocoumafen and in which the flocoumafen is predominantly in the form of said enantiomer E₂.

The invention thus also relates to a composition according to the invention comprising flocoumafen, said diastereoisomer D_{2,3} of flocoumafen of the composition being optically active. However, it is not excluded for the flocoumafen of the composition according to the invention to be optically inactive.

The invention also relates to the use of a composition according to the invention for the preparation of a rodenticidal bait for target rodent pests.

The invention also relates to a rodenticidal bait comprising a composition according to the invention, and at least one excipient that is edible for target rodent pests.

A rodenticidal bait according to the invention comprises:

at least one excipient that is edible for target rodent pests; and

a configurational stereoisomer, named enantiomer E₂, of flocoumafen, with the exclusion of a racemic mixture of said enantiomer E₂ and of a configurational stereoisomer, named enantiomer E₃, of flocoumafen;

said enantiomer E₂ having, by chromatographic analysis of a flocoumafen composition comprising four configurational stereoisomers of flocoumafen performed under the conditions described below, a retention time t₂;

said enantiomer E₃ having, by chromatographic analysis of a flocoumafen composition comprising four configurational stereoisomers of flocoumafen performed under these same conditions, a retention time t₃; t₂ and t₃ being values such that t₁<t₂<t₃<t₄; t₁ and t₄ representing the retention times of each of the configurational stereoisomers of flocoumafen different from said enantiomer E₂ and from said enantiomer E₃, said analysis being performed at a temperature of 23.5° C. and under the following conditions:

on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;

using, as liquid mobile phase, a mixture formed from acetonitrile (A) and water comprising 0.1% by volume of formic acid (B), with an AB volume ratio of 92/8 and with a flow rate of the liquid mobile phase in the chromatography column of 0.25 mL/minute;

by injection into the chromatography column of a volume of 1 μL of flocoumafen composition at a concentration of 1 μg of flocoumafen per millilitre of acetonitrile.

Advantageously, a rodenticidal bait according to the invention comprises an excipient that is edible for target rodent pests and flocoumafen in which the amount of said enantiomer E₂ is greater than the amount of said enantiomer E₃. Advantageously, it may also be a rodenticidal bait comprising flocoumafen and in which the flocoumafen is predominantly in the form of said enantiomer E₂.

The inventors who succeeded in separating the enantiomers of flocoumafen and in isolating said enantiomer E₂ also discovered, entirely surprisingly and unexpectedly, that the configurational stereoisomers of flocoumafen do not all have the same persistence in the liver of rodents which consume flocoumafen and that said enantiomer E₂ in fact has a persistence in the liver of rodents which consume a rodenticidal bait according to the invention that is less than the persistence of enantiomer E₄ of said diastereoisomer D_{1,4}, but also less than the hepatic persistence of flocoumafen.

They observed that, when it is ingested by a target rodent pest, said enantiomer E₂ is less persistent and disappears from the liver of the target rodent pest which has consumed a bait according to the invention more rapidly than the disappearance of enantiomer E₄ of said diastereoisomer D_{1,4} and than the disappearance of flocoumafen, the amount of said enantiomer E₂ being, however, rodenticidal.

The inventors observed that said enantiomer E₂, although having a persistence in the liver of target rodent pests which is lower than the persistence of enantiomer E₄ and lower than the persistence of flocoumafen, in fact makes it possible, and in a manner that is entirely unexplained to date, to efficiently control target rodent pests.

The residual amount of said enantiomer E₂ in the body of a rodent poisoned with a rodenticidal bait according to the invention decreases more rapidly than the amount of enantiomer E₄ of said diastereoisomer D_{1,4} and than the amount of flocoumafen. The dead or live target rodent pest which has ingested said enantiomer E₂ is, as a result, less dangerous with respect to non-rodent mammals and birds which consume the target rodent pest—dead or alive—and in particular with respect to predators (especially non-rodent mammals and birds) which preferentially consume the viscera of their prey and in particular their liver. Said enantiomer E₂ is thus sparingly toxic to the environment.

A rodenticidal bait according to the invention is able to be used to control populations of target rodent pests that are resistant to known rodenticidal treatments.

Advantageously, a rodenticidal bait according to the invention comprises a mass amount of flocoumafen such that the ratio of this mass amount of flocoumafen to the mass amount of rodenticidal bait is less than 200 ppm, i.e. less than 200 mg of flocoumafen per kilogram of rodenticidal bait. Advantageously, it comprises a mass amount of flocoumafen such that the ratio of this mass amount of flocoumafen to the mass amount of rodenticidal bait is greater than 1 ppm. Advantageously, a rodenticidal bait according to the invention comprises a mass amount of flocoumafen such that the ratio of this mass amount of flocoumafen to the mass amount of rodenticidal bait is between 1 ppm and 200 ppm (1 mg to 200 mg of flocoumafen per kilogram of rodenticidal bait). Advantageously, the ratio of this mass amount of flocoumafen to the mass amount of rodenticidal bait is between 1 ppm and 100 ppm (1 mg to 100 mg of flocoumafen per kilogram of rodenticidal bait), especially between 1 ppm and 50 ppm (1 mg to 50 mg of flocoumafen per kilogram of rodenticidal bait), preferably between 10 ppm and 25 ppm (10 mg to 25 mg of flocoumafen per kilogram of rodenticidal bait).

Advantageously, a rodenticidal bait according to the invention comprises an amount of said enantiomer E₂ such that the ratio of this amount to the amount of flocoumafen in the rodenticidal bait is greater than 70%, more particularly greater than 80%, preferably greater than 90%, particularly preferentially greater than 95%, more preferentially greater than 98%, even more preferentially greater than 99% or about 100%, and of flocoumafen in a mass proportion of less than 100 ppm—especially less than 50 ppm—relative to the rodenticidal bait.

Advantageously and according to the invention, the excipient that is edible for target rodent pests is chosen to allow consumption of the rodenticidal bait by target rodent pests. Advantageously and according to the invention, each edible excipient is non-lethal to target rodent pests. The edible excipient is not in itself rodenticidal.

Advantageously and according to the invention, the edible excipient comprises at least one food chosen from the group formed from cereal seeds—especially hulled cereal seeds—cereal seed meals, cereal seed flours, cereal seed flakes, cereal bran and non-cereal seeds, for example alfalfa seeds—especially in hulled form, in the form of meal, in the form of flour, or in the form of flakes or bran. The edible excipient may comprise any support that can be consumed by target rodent pests.

Advantageously, the edible excipient comprises at least one food chosen from the group formed from foods of plant origin and foods of animal origin. Advantageously, the edible excipient comprises at least one food chosen to be able to stimulate the appetite of the target rodent pests. In particular, this food is chosen from the group formed from seeds of one or more cereals, hulled seeds of one or more cereals, meals of seeds of one or more cereals, flakes of seeds of one or more cereals, bran of one or more cereals and flour of seeds of one or more cereals. By way of example, the cereals are chosen from the group formed from oat, wheat, barley, corn, soybean and rice.

Advantageously, the food is chosen from the group formed from sweetened foods. For example, they may be foods comprising at least one sugar chosen from the group formed from sucrose, lactose, fructose and glucose. It may be a sugar syrup—for example a sugar syrup obtained by hydrolysis of starch—or a sugar syrup obtained by hydrolysis of sucrose (invert sugar syrup), or a beet sugar syrup, or a maple syrup or a sugarcane syrup, or a syrup obtained from a plant of the Stevia genus.

Advantageously, the food is chosen from the group formed from coconut albumen (copra) flakes and flour. Advantageously, the food is chosen from the group formed from walnuts, hazelnuts and almonds—in grated and/or powder form.

Advantageously, the food is chosen from the group formed from plant fats, plant oils (for example rapeseed oil, soybean fat, sunflower oil, cocoa butter, groundnut oil, groundnut butter, corn oil, palm oil), animal fats and animal oils (butter, lard, fish oil).

Advantageously, the food is chosen from the group formed from proteins of plant origin and proteins of animal origin. By way of example, examples that may be mentioned include powdered milk—especially powdered skimmed milk—eggs—especially powdered eggs—protein hydrolysates of animal origin and protein hydrolysates of plant origin.

Advantageously and according to the invention, the rodenticidal bait is chosen from the group formed from solid baits comprising flocoumafen and a solid edible excipient. Advantageously, the rodenticidal bait is a solid in divided form, for example in the form of balls or granules. Advantageously, the rodenticidal bait may be a solid in block or paste form that may be consumed by the target rodent pests or a solid material that may be nibbled by the target rodent pests. Advantageously, the solid rodenticidal bait according to the invention may be in the form of a rigid block, a semi-rigid block, a foam, a powder or a gel.

Advantageously, the rodenticidal bait which is in the form of a powder, in the form of a foam or in the form of a gel is suitable for soiling the fur of the target rodent pest(s) and for being ingested by said pest(s) during their grooming.

It may be a solid rodenticidal bait comprising flocoumafen, with the exclusion of a racemic mixture of said enantiomer E₂ and of said enantiomer E₃. It may also be a solid rodenticidal bait comprising flocoumafen and in which the amount of said enantiomer E₂ is greater than the amount of said enantiomer E₃. It may also be a solid rodenticidal bait comprising flocoumafen in which the flocoumafen is predominantly in the form of said enantiomer E₂.

Advantageously and according to the invention, the rodenticidal bait is chosen from the group formed from liquid baits comprising flocoumafen and a liquid edible excipient. The rodenticidal bait is then a drink for target rodent pests. It may be a solution of flocoumafen in a solvent for flocoumafen, with the exclusion of a racemic mixture of said enantiomer E₂ and of said enantiomer E₃. It may also be a solution of flocoumafen in a solvent for flocoumafen and in which the amount of said enantiomer E₂ is greater than the amount of said enantiomer E₃. It may also be a solution of flocoumafen in a solvent for flocoumafen and in which the flocoumafen is predominantly in the form of said enantiomer E₂.

It may also be a suspension of flocoumafen in solid form in a liquid medium. It may also be an emulsion of flocoumafen in a liquid medium.

The invention thus also relates to a rodenticidal bait comprising said enantiomer E₂, with the exclusion of a racemic mixture of said enantiomer E₂ and of said enantiomer E₃, the flocoumafen of the rodenticidal bait being optically active. However, it is not excluded for the flocoumafen of the rodenticidal bait according to the invention comprising said enantiomer E₂, with the exclusion of a racemic mixture of said enantiomer E₂ and of said enantiomer E₃, to be optically inactive.

Advantageously, the rodenticidal bait comprises at least one dye. Such a dye makes it possible in particular to give said rodenticidal bait a colour that is readily detectable and identifiable by a person handling the rodenticidal bait.

Advantageously, the rodenticidal bait comprises at least one preserving agent capable of ensuring its conservation during its storage. Advantageously, the rodenticidal bait comprises at least one bittering compound such as denatonium benzoate, also known as Bitrex®, which is intended to reduce the risks of accidental consumption by non-target organisms.

Advantageously, in a particular variant, the composition and the rodenticidal bait according to the invention exclusively comprise flocoumafen, with the exclusion of a racemic mixture of the enantiomers of said diastereoisomer D_{2,3}, as rodenticidal substance. In particular, the composition and the rodenticidal bait according to the invention are free of any other anticoagulant substance for rodenticidal use different from flocoumafen. However, in this variant according to the invention, the composition and the rodenticidal bait may comprise any pest-control substance other than a rodenticide, such as an insecticidal and/or acaricidal substance.

Advantageously, in another particular variant, the composition and the rodenticidal bait according to the invention comprise flocoumafen, with the exclusion of a racemic mixture of said enantiomer E₂ and of said enantiomer E₃, and at least one other substance different from flocoumafen as rodenticidal substance. This other rodenticidal substance different from flocoumafen may be another anticoagulant substance—especially of the anti-vitamin K type or not—or any other non-anticoagulant rodenticidal substance.

The invention also relates to a process for controlling target rodent pests, in which there is spread an amount of rodenticidal bait comprising:

at least one excipient that is edible for target rodent pests; and

a configurational stereoisomer, named enantiomer E₂, of flocoumafen, with the exclusion of a racemic mixture of said enantiomer E₂ and of a configurational stereoisomer, named enantiomer E₃, of flocoumafen;

said enantiomer E₂ having, by chromatographic analysis of a flocoumafen composition comprising four configurational stereoisomers of flocoumafen performed under the conditions described below, a retention time t₂;

said enantiomer E₃ having, by chromatographic analysis of a flocoumafen composition comprising four configurational stereoisomers of flocoumafen performed under these same conditions, a retention time t₃; t₂ and t₃ being values such that t₁<t₂<t₃<t₄; t₁ and t₄ representing the retention times of each of the configurational stereoisomers of flocoumafen different from said enantiomer E₂ and from said enantiomer E₃, said analysis being performed at a temperature of 23.5° C. and under the following conditions:

on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;

using, as liquid mobile phase, a mixture formed from acetonitrile (A) and water comprising 0.1% by volume of formic acid (B), with an AB volume ratio of 92/8 and with a flow rate of the liquid mobile phase in the chromatography column of 0.25 mL/minute;

by injection into the chromatography column of a volume of 1 μL of flocoumafen composition at a concentration of 1 μg of flocoumafen per millilitre of acetonitrile.

The invention also relates to a process for controlling target rodent pests, in which there is spread an amount of rodenticidal bait according to the invention, said amount of bait being sufficient to be rodenticidal.

In such a process, advantageously and according to the invention, the amount of said enantiomer E₂ in the rodenticidal bait is greater than the amount of said enantiomer E₃ in the rodenticidal bait. Said diastereoisomer D₂₃ is predominantly in the form of said enantiomer E₂. In such a process, advantageously and according to the invention, flocoumafen is predominantly in the form of said enantiomer E₂.

Advantageously and as a variant according to the invention, the following are chosen in combination:

the edible excipient;

an amount of said enantiomer E₂ different from the amount of said enantiomer E₃ in the rodenticidal bait;

a proportion of said enantiomer E₂ relative to the flocoumafen;

a mass proportion of flocoumafen in the rodenticidal bait; and

an amount of spread rodenticidal bait;

so that target rodent pests consume an amount of flocoumafen that is sufficient to be lethal to said target rodent pests which consume said rodenticidal bait in the course of a single period of 24 consecutive hours.

A rodenticidal bait according to this variant of the invention is a rodenticidal bait that is mortal in a single intake, or a “one-shot” bait. Advantageously and according to this variant of the invention, the mass proportion of flocoumafen in the rodenticidal bait is less than 200 ppm, especially between 2 ppm and 200 ppm, preferably between 2 ppm and 100 ppm, more preferentially between 2 ppm and 50 ppm and in particular between 15 ppm and 50 ppm.

Advantageously and in another variant according to the invention, the following are chosen in combination:

the edible excipient;

an amount of said enantiomer E₂ different from the amount of said enantiomer E₃ in the rodenticidal bait;

a proportion of said enantiomer E₂ relative to the flocoumafen;

a mass proportion of flocoumafen in the rodenticidal bait; and

an amount of spread rodenticidal bait;

so that target rodent pests consume an amount of flocoumafen:

which is non-lethal to target rodent pests, i.e. which is generally non-lethal to target rodent pests which consume said rodenticidal bait over a period of 24 consecutive hours; and

which is sufficient to be lethal to target rodent pests which consume said rodenticidal bait over several 24-hour periods, said periods being consecutive.

This other variant of the invention is thus also directed towards a process for controlling target rodent pests, in which there is spread an amount of rodenticidal bait that is lethal for target rodent pests which durably consume this rodenticidal bait and generally non-lethal for non-target rodents or animals which accidentally consume this rodenticidal bait. This is then referred to as a “multi-dose” or “multi-feeding” control process. In such a process according to the invention, the consumption of rodenticidal bait by a target rodent pest over a period of 24 hours is insufficient to result in the death of said rodent, whereas repeated consumption of rodenticidal bait over at least two consecutive days can result in the death of the target rodent pest.

This other variant of the invention is thus directed towards a process for controlling a population of target rodent pests, in which target rodent pests are provided with an amount of rodenticidal bait that is liable to be ingested by the target rodent pests, said amount of rodenticidal bait being sufficient to kill target rodent pests which consume said rodenticidal bait over several days.

Advantageously, in this other variant of a process according to the invention, the amount of rodenticidal bait spread, the mass proportion of flocoumafen in the rodenticidal bait and the proportion of said enantiomer E₂ relative to said diastereoisomer D₂₃ are adapted so that the consumption of the rodenticidal bait is lethal to target rodent pests which daily consume rodenticidal bait over at least two 24-hour periods, especially from 3 to 7 periods, said periods being consecutive.

Advantageously, in this other variant of a process according to the invention, since the proportion of said enantiomer E₂ is greater than 95%—especially about 100%—relative to the flocoumafen in the rodenticidal bait, the mass proportion of flocoumafen relative to the rodenticidal bait is between 2 ppm and 100 ppm, especially between 2 ppm and 50 ppm, in particular between 2 ppm and 15 ppm, especially about 10 ppm.

In a process according to the invention, target rodent pests are provided with an amount of rodenticidal bait that is sufficient to satisfy the daily appetite of the target rodent pests, said rodenticidal bait predominantly comprising said enantiomer E₂ relative to said diastereoisomer D₂₃ and relative to the flocoumafen.

In a process according to the invention, the amount of rodenticidal bait spread, the ratio of the amount of said enantiomer E₂ to the amount of said diastereoisomer D_{2,3}, the ratio of the amount of said enantiomer E₂ to the amount of flocoumafen and the mass proportion of flocoumafen relative to the rodenticidal bait are adapted so as to allow consumption of rodenticidal bait for several days by target rodent pests, while at the same time limiting:

the risks of primary intoxication of non-target mammals and birds which are liable to consume such a rodenticidal bait only occasionally and accidentally;

the risks of secondary intoxication, for example of predators of target rodents, which are liable to consume target rodents—dead or live—that have ingested an amount of said bait.

The invention also relates to a configurational stereoisomer of flocoumafen, to a process for obtaining this configurational stereoisomer, to a composition and a rodenticidal bait and to a process for controlling target rodent pests, which are characterized in combination by all or some of the characteristics mentioned hereinabove or hereinbelow.

Other aims, characteristics and advantages of the invention will emerge on reading the following description and the examples, which are given for purely non-limiting purposes and which refer to the attached figures, in which:

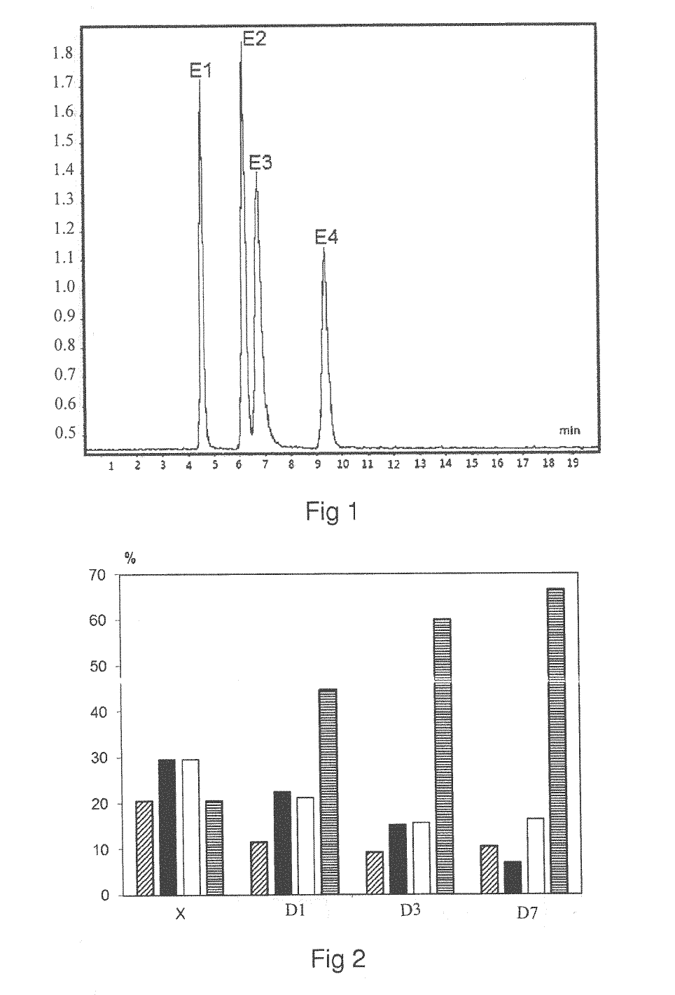

FIG. 1 is a representative chromatogram of the separation of the configurational stereoisomers of flocoumafen on a chiral column;

FIG. 2 is a histogram representation of the persistence of the configurational stereoisomers of flocoumafen in rat liver.

Analysis of the Configurational Stereoisomers of Flocoumafen

The configurational stereoisomers of flocoumafen are separated by high-pressure liquid chromatography using a LUX® Cellulose-4 chiral column (00F-4490-B0, Phenomenex, Le Pecq, France) as stationary phase and a mixture formed from acetonitrile (A) and water comprising 0.1% by volume of formic acid (B), with an AB volume ratio of 92/8, as mobile phase with a flow rate of the liquid mobile phase in the chromatography column of 0.25 mL/minute. 1 μL of extract to be analysed at a concentration of 1 μg/mL of acetonitrile is injected. Detection is performed by tandem mass spectrometry (MS/MS) in negative electrospray ionization (ESI) mode. The temperature of the nebulizer gas is 350° C. and its flow rate is 8 L/minute. The pressure of the nebulizer gas is brought to 2700 hPa. In particular, the MRM (“Multiple Reaction Monitoring”) transitions m/z 541.1→382.1 and m/z 541.1→161, corresponding to the flocoumafen signal, are analysed.

The retention time values for each of the flocoumafen enantiomers are, under the conditions described:

t₁=4.5 min for enantiomer E₁ of said diastereoisomer D_{1,4};

t₂=6.2 min for said enantiomer E₂ according to the invention;

t₃=6.8 min for said enantiomer E₃ of said diastereoisomer D_{2,3}; and

t₄=9.3 min for enantiomer E₄ of said diastereoisomer D_{1,4}.

The retention time values t₁, t₂, t₃ and t₄ are liable to vary, especially with the temperature of the chromatography column. However, under these chromatographic conditions, the order of elution of the flocoumafen enantiomers remains unchanged. As a guide, the retention time value (t₁) of enantiomer E₁ of said diastereoisomer D₁₄ may range between 4.4 min and 4.6 min. The retention time value (t₂) of enantiomer E₂ of said diastereoisomer D_{2,3} may range between 5.9 min and 6.4 min. The retention time value (t₃) of enantiomer E₃ of said diastereoisomer D₂₃ may range between 6.4 min and 6.9 min. The retention time value (t₄) of said enantiomer E₄ according to the invention may range between 8.9 min and 9.4 min.

Extraction of Flocoumafen from the Liver of Rats Tube-Fed with Flocoumafen

Homogenization of the Liver Sample

About 0.525 g (±0.025 g) of rat liver is weighed out accurately and placed in a 50 mL polypropylene tube. 10 mL of acetone are added and the suspension is homogenized using an Ultra-Turrax® homogenizer/disperser for a time of about 30 seconds. The homogenizer/disperser shaft is rinsed with hot water and then twice with 20 mL of acetone in a polypropylene tube. The homogenate is centrifuged for 5 minutes at a centrifugation speed of 3000 rpm (revolutions per minute). The supernatant is collected and transferred into a test tube. The sample is subjected to evaporation under a stream of nitrogen (N₂) at a temperature of 40° C. so as to form a dry extract.

Lipid Removal

1 mL of acetonitrile is added to the tube containing the dry extract so as to dissolve it. The acetonitrile solution is washed twice successively with 1 mL of hexane. The lipid-free extract is dried under a stream of nitrogen (N₂) at a temperature of 40° C. and is then taken up in 0.5 mL of methanol and dissolved by vortex stirring. 0.5 mL of ultra-pure (Milli-Q) water is then added. The sample is vortex-homogenized.

Solid-Phase Extraction (SPE) of Flocoumafen

1 mL of dichloromethane (CH₂Cl₂), then 1 mL of methanol (CH₃OH), then 1 mL of ultra-pure (Milli-Q) water are passed through an Oasis HLB 1 cc cartridge (WAT094225, Waters). The lipid-free liver extract (1 mL MeOH/Milli-Q H₂O) containing flocoumafen is then loaded onto the top of the preconditioned cartridge. The liver extract penetrates through the cartridge by gravity on contact with the solid phase of the cartridge. 1 mL of washing solution formed from methanol (CH₃OH) and ultra-pure water (H₂O) in a 90/10 volume proportion is loaded onto the top of the cartridge. The cartridge is dried by suction under vacuum connected to the bottom of the cartridge. 1 mL of eluting solution formed from dichloromethane (CH₂Cl₂) and methanol (CH₃OH) in a 90/10 volume proportion is then loaded onto the top of the cartridge and an eluate comprising flocoumafen is collected at the bottom of the cartridge. The solvent of the eluate is evaporated off under a stream of nitrogen (N₂) at a temperature of 40° C. The sample is taken up in 0.5 mL of acetonitrile (NC—CH₃) and the acetonitrile solution containing flocoumafen is filtered through a 0.2 μm porosity filter.

Hepatic Persistence of the Configurational Stereoisomers of Flocoumafen in Rats

A solution comprising flocoumafen in a mixture of vegetable oil and 5% of DMSO so that the amount of flocoumafen ingested by each rat is about 2.3 mg per kilogram of rat is administered orally (per os) to 8-week-old male and female laboratory rats (Sprague-Dawley rats, Charles River, Saint-Germain-sur-l'Arbresle, France) weighing between 180 and 200 g. The tube-fed rats are treated daily by subcutaneous administration of a dose of vitamin K1 at a rate of 1 U per rat so as to keep the rats alive throughout the experiment. The ratio of the sum of the amounts of said enantiomer E₂ and of said enantiomer E₃ (diastereoisomer D_{2,3}) to the amount of flocoumafen in the tube-feeding solution is 59% and the ratio of the sum of the amounts of said enantiomer E₁ and of said enantiomer E₄ (diastereoisomer D_{1,4}) to the amount of flocoumafen in the tube-feeding solution is 41%.

At 1 day (D+1), 3 days (D+3) and 7 days (D+7) after tube-feeding, three male rats and three female rats anaesthetized beforehand with isoflurane are euthanized, the liver of the euthanized rats is removed and the amounts of each of the configurational stereoisomers of flocoumafen present in the liver of the tube-fed male and female rats are then extracted from the liver and assayed. The results obtained on the male rats are given in table 1 below and the results obtained on the female rats are given in table 2 below in which the values of the concentration of each of the configurational stereoisomers of flocoumafen in the liver are the mean of the values measured on three rats expressed in nanograms (ng) per gram of liver.

TABLE 1
(male rats)

| Hepatic concentration, ng/g | D + 1 | D + 3 | D + 7 |
|---|---|---|---|
| Enantiomer E₁ | 196 | 47 | 0 |
| Enantiomer E₂ | 3904 | 1962 | 319 |
| Enantiomer E₃ | 3301 | 1862 | 647 |
| Enantiomer E₄ | 8143 | 9906 | 2623 |

TABLE 2
(female rats)

| Hepatic concentration, ng/g | D + 1 | D + 3 | D + 7 |
|---|---|---|---|
| Enantiomer E₁ | 4585 | 2692 | 1196 |
| Enantiomer E₂ | 5396 | 2522 | 469 |
| Enantiomer E₃ | 5478 | 2734 | 1228 |
| Enantiomer E₄ | 10342 | 7765 | 5054 |

FIG. 2 represents the change in the percentage of the mass amount of each enantiomer (E₁ and E₄) of diastereoisomer D_{1,4} and of each enantiomer (E₂ and E₃) of diastereoisomer D_{2,3} relative to the amount of flocoumafen retained in the liver of the tube-fed rats (male and female) euthanized on D+1, D+3 and D+7. The percentage of said enantiomer E₁ is represented by oblique-hatched columns, the percentage of said enantiomer E₄ is represented by horizontally-hatched columns, the percentage of said enantiomer E₂ is represented by black columns and the percentage of said enantiomer E₃ is represented by white columns. The respective proportions of each of the enantiomers (E₁ and E₄) of diastereoisomer D_{1,4} and of the enantiomers (E₂ and E₃) of diastereoisomer D_{2,3} in the flocoumafen of the tube-feeding composition are represented in column “X” FIG. 2 and are 20.5% for enantiomer E₁, 20.5% for enantiomer E₄, 29.5% for enantiomer E₂ of diastereoisomer D_{2,3} and 29.5% for enantiomer E₃ of diastereoisomer D₂₃.

The persistence of said enantiomer E₂ in the liver of the male and female rats is lower than the persistence of enantiomer E₄ of said diastereoisomer D_{1,4} and lower than the persistence of flocoumafen in the liver of the rats. Said enantiomer E₂ of flocoumafen is thus able to be used as a rodenticidal substance which has reduced toxicity to the environment.

It goes without saying that the invention may be the subject of numerous implementation variants and applications. In particular, a composition, a rodenticidal bait and a process for controlling target rodent pests are subject to an infinite number of variants both in the formulation of the rodenticidal bait and in the embodiments of the process.

1-11. (canceled)

12. Configurational stereoisomer, named enantiomer E₂, of flocoumafen, said enantiomer E₂ having, by chromatographic analysis of a flocoumafen composition comprising four configurational stereoisomers of flocoumafen performed under the conditions described below, a retention time t₂ having a value such that t₁<t₂<t₃<t₄; t₁, t₃ and t₄ representing the retention times of the configurational stereoisomers of flocoumafen different from said enantiomer E₂, the chromatographic analysis being performed at a temperature of 23.5° C. and under the following conditions:
on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;
using, as liquid mobile phase, a mixture formed from acetonitrile (A) and water comprising 0.1% by volume of formic acid (B), with an A/B volume ratio of 92/8 and with a flow rate of the liquid mobile phase in the chromatography column of 0.25 mL/minute;
by injection into the chromatography column of a volume of 1 μL of flocoumafen composition at a concentration of 1 μg of flocoumafen per millilitre of acetonitrile.

13. Composition comprising a configurational stereoisomer, named enantiomer E₂, of flocoumafen, with the exclusion of a racemic mixture of said enantiomer E₂ and of a configurational stereoisomer, named enantiomer E₃, of flocoumafen;
said enantiomer E₂ having, by chromatographic analysis of a flocoumafen composition comprising four configurational stereoisomers of flocoumafen performed under the conditions described below, a retention time t₂;
said enantiomer E₃ having, by analysis of flocoumafen comprising four configurational stereoisomers of flocoumafen performed under these same conditions, a retention time t₃; t₂ and t₃ being values such that t₁<t₂<t₃<t₄; t₁ and t₄ representing the retention times of each of the configurational stereoisomers of flocoumafen different from said enantiomer E₂ and from said enantiomer E₃, the chromatographic analysis being performed at a temperature of 23.5° C. and under the following conditions:
on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;
using, as liquid mobile phase, a mixture formed from acetonitrile (A) and water comprising 0.1% by volume of formic acid (B), with an A/B volume ratio of 92/8 and with a flow rate of the liquid mobile phase in the chromatography column of 0.25 mL/minute;
by injection into the chromatography column of a volume of 1 μL of flocoumafen composition at a concentration of 1 μg of flocoumafen per millilitre of acetonitrile.

14. Composition according to claim 13, wherein the amount of said enantiomer E₂ is greater than the amount of said enantiomer E₃ in the composition.

15. Composition according to claim 13, wherein the flocoumafen is predominantly in the form of said enantiomer E₃ in the composition.

16. Composition according to claim 13, comprising an amount of said enantiomer E₂ such that the ratio of this amount to the amount of flocoumafen in the composition is greater than 25%.

17. Composition according to claim 13, comprising an amount of said enantiomer E₂ such that the ratio of this amount to the amount of flocoumafen in the composition is greater than 95%.

18. Rodenticidal bait comprising a composition according to claim 13 and at least one excipient that is edible for target rodent pests.

19. Bait according to claim 18, wherein the edible excipient comprises at least one food chosen from the group formed from cereal seeds, cereal seed meals, cereal seed flours, cereal seed flakes, cereal bran and non-cereal seeds.

20. Bait according to claim 18, comprising a mass amount of flocoumafen such that the ratio of this mass amount of flocoumafen to the mass amount of rodenticidal bait is less than 200 ppm.

21. Process for controlling target rodent pests, in which there is spread an amount of rodenticidal bait comprising:
at least one excipient that is edible for target rodent pests,
a configurational stereoisomer, named enantiomer E₂, of flocoumafen, with the exclusion of a racemic mixture of said enantiomer E₂ and of a configurational stereoisomer, named enantiomer E₃, of flocoumafen;
said enantiomer E₂ having, by chromatographic analysis of a flocoumafen composition comprising four configurational stereoisomers of flocoumafen performed under the conditions described below, a retention time t₂;
said enantiomer E₃ having, by chromatographic analysis of a flocoumafen composition comprising four configurational stereoisomers of flocoumafen performed under these same conditions, a retention time t₃;
t₂ and t₃ being values such that t₁<t₂<t₃<t₄; t₁ and t₄ representing the retention times of each of the configurational stereoisomers of flocoumafen different from said enantiomer E₂ and from said enantiomer E₃, said analysis being performed at a temperature of 23.5° C. and under the following conditions:
on a high-pressure liquid chromatography column of dimensions 150×2 mm, and comprising a chiral stationary phase constituted of particles of tris(4-chloro-3-methylphenyl carbamate) cellulose, said particles having a mean size of 3 μm and having a mean pore size of 1000 Å;
using, as liquid mobile phase, a mixture formed from acetonitrile (A) and water comprising 0.1% by volume of formic acid (B), with an A/B volume ratio of 92/8 and with a flow rate of the liquid mobile phase in the chromatography column of 0.25 mL/minute;
by injection into the chromatography column of a volume of 1 μL of flocoumafen composition at a concentration of 1 μg of flocoumafen per millilitre of acetonitrile.

22. Chromatographic process for obtaining said enantiomer E₂ according to claim 12, in which:
a high-pressure liquid chromatography column of dimensions 150×2 mm, and comprising a chiral stationary phase constituted of particles of tris(4-chloro-3-methylphenyl carbamate) cellulose, is chosen, said particles having a mean size of 3 μm and having a mean pore size of 1000 Å;
a mixture formed from acetonitrile (A) and water comprising 0.1% by volume of formic acid (B), with an A/B volume ratio of 92/8 and with a flow rate of the liquid mobile phase in the chromatography column of 0.25 mL/minute, is chosen as liquid mobile phase;
separation of the configurational stereoisomers of flocoumafen is performed at room temperature, during which:
a liquid composition comprising said enantiomer E₂ is introduced into the top of the chromatography column; and then
the liquid composition is entrained with the mobile phase in the chromatography column under conditions suitable for separating the configurational stereoisomers of flocoumafen, and a fraction of the mobile phase comprising said enantiomer E₂ with a retention time t₂ having a value such that t₁<t₂<t₃<t₄; t₁, t₃ and t₄ representing the retention times of each of the configurational stereoisomers of flocoumafen different from said enantiomer E₂, is collected separately from said enantiomer E₃ of retention time t₃; and
the liquid mobile phase of said fraction is removed so as to obtain said enantiomer E₂.

23. Composition according to claim 14, wherein the flocoumafen is predominantly in the form of said enantiomer E₃ in the composition.

24. Composition according to claim 14, comprising an amount of said enantiomer E₂ such that the ratio of this amount to the amount of flocoumafen in the composition is greater than 25%.

25. Composition according to claim 15, comprising an amount of said enantiomer E₂ such that the ratio of this amount to the amount of flocoumafen in the composition is greater than 25%.

26. Composition according to claim 14, comprising an amount of said enantiomer E₂ such that the ratio of this amount to the amount of flocoumafen in the composition is greater than 95%.

27. Composition according to claim 15, comprising an amount of said enantiomer E₂ such that the ratio of this amount to the amount of flocoumafen in the composition is greater than 95%.

28. Composition according to claim 16, comprising an amount of said enantiomer E₂ such that the ratio of this amount to the amount of flocoumafen in the composition is greater than 95%.

29. Rodenticidal bait comprising a composition according to claim 14 and at least one excipient that is edible for target rodent pests.

30. Rodenticidal bait comprising a composition according to claim 15 and at least one excipient that is edible for target rodent pests.

31. Rodenticidal bait comprising a composition according to claim 16 and at least one excipient that is edible for target rodent pests.
